(19) United States
(12) Patent Application Publication
(10) Pub. No.: US 2024/0416045 A1
(43) Pub. Date: Dec. 19, 2024

(54) A SYRINGE

(71) Applicant: MEDTECH CONCEPT LLC, Lake Worth, FL (US)

(72) Inventor: Patrick MARTINSSON, Malta (MT)

(21) Appl. No.: 18/702,348

(22) PCT Filed: Oct. 18, 2022
(86) PCT No.: PCT/US2022/046935

§ 371 Date: Apr. 18, 2024

(30) Foreign Application Priority Data
Oct. 19, 2021 (SE) 2100156-5

(51) Int. Cl. A61M 5/315

(52) U.S. Cl. CPC A61M 5/31513; A61M 2202/30; A61M 2205/02

(57) ABSTRACT

A syringe having a front portion carrying a hollow, axially extending needle, a container, running from said front portion and forming a container rear end portion, a plunger, extending and being movable axially in the container, the needle rear end being in fluid connection with the container and the: plunger being arranged to suck liquid, through the needle when drawn away from the needle and to discharge liquid through the needle when pushed towards the needle, the plunger having a piston portion, turned to the needle rear end, and a coupling portion arranged to co-act with the piston portion for moving the piston portion in the container. The syringe is especially characterized in that the container wall is made of a material having a certain resiliency and an inner diameter smaller than the outer diameter of the piston portion, such that a sealing action appears between the container and the piston portion.

### TECHNICAL FIELD

The present invention relates to a syringe according to the introductory portion of the attached claim 1.

### BACKGROUND

A general problem associated with syringes, especially those intended for injecting substances, e.g. a liquid comprising insulin, glycagone or a vaccine, into the human body, is that it is normally preferable that they are intended for single injection purposes, this being for contamination reasons. Thus, well known are the extremely serious problems associated with re-use of syringes as a source of spread of infections.

### PRIOR ART

Therefore, there are a great number of prior art syringes being designed to prevent re-use, these designs being, however, complicated, expensive to manufacture and complicated to mount.

The object of the present invention is to provide a syringe less associated with the problems related to prior art syringes and yet, in principle, avoiding re-use possibilities.

### SUMMARY OF THE INVENTION

These and other objects are obtained by a syringe according to what is specified in independent claim 1.

Further objects are obtained by what is specified in the dependent claims.

Thus, according to preferred embodiments the syringe according to the present invention has a front portion carrying a hollow, axially extending needle, a container, a barrel, running from said front portion and forming a container rear end portion, a plunger, extending and being movable axially in the container, the needle rear end being in fluid connection with the container and the plunger being arranged to suck liquid, e.g. a liquid comprising insulin, glycagon, a vaccine or other substances, through the needle when drawn away from the needle and to discharge liquid through the needle when pushed towards the needle, the plunger having a piston portion, turned to the needle rear end, the plunger length in relation to the container length being adapted so that when the syringe is arranged in a delivery state a certain portion of the plunger extending beyond the container rear end portion, the container wall being of a cylindrically shaped pipe like body of a material having a certain resiliency and an inner diameter smaller than the outer diameter of the piston portion, such that the container is widened by the piston portion and such that a sealing action appears between the inner wall of the container and the peripheral surface of the piston portion due to a pressure excerted by the widened container wall.

The general design of syringes according to the present invention characterized in very few parts will make manufacturing easier and cheaper.

According to preferred embodiments the container is made of a polymeric material, so as to obtain a desired resilience and sealing properties of the container wall.

Further according to preferred embodiments the needle is carried by a connection portion arranged to be connected to a container front portion by means of a liquid tight thread arrangement.

Further preferred is that the plunger has a front connection portion arranged to fit into a rear opening of the piston portion for connection.

Preferred is also that the plunger has a cross like cross section

Further preferred is that the syringe comprises a grip portion having extensions for holding the syringe during injection by the syringe, the grip portion preferably being fastened onto the piston by a snap arrangement.

Still further preferred is that the syringe according to the present invention is adapted to contain a single vaccine dose, normally about one milliliter.

Further, according to preferred embodiments, the size of the syringe is adapted to the kind of liquid to be injected, e.g. the concentration of the active substance of the liquid, which means that if a larger volume of liquid is desired and therefore a bigger size of the syringe is necessary for obtaining a desired effect of a certain injection.

According to preferred embodiments the syringe is adapted to injection of a preferred kind of vaccine for people. Single injection use will dramatically decrease infection due to vast vaccination campaigns.

According to preferred embodiments the syringe is substantially axially symmetrical. This makes the syringe easy to handle and to manufacture.

### BRIEF DESCRIPTION OF THE DRAWINGS

A better understanding of the present invention may be had by considering the following detailed description in conjunction with the attached drawings, in which like details are designated with like designations and in which:

FIG. 1 diagrammatically, in a sectional view, shows a according to the present invention seen perpendicularly to the axial direction of the syringe;

FIG. 2 diagrammatically shows, in two different projections, a piston portion according to the present invention and a grip shown in two different views;

FIG. 3 diagrammatically shows, in four different states of use a)-d), wherein a) corresponds to an unused state, b) corresponds to a liquid filled state, c) corresponds to a ready to inject state and d) corresponds to a used and locked state;

c

FIG. 4 diagrammatically, in three sectional views a)-c), different syringe needle arrangements; and

FIG. 5 diagrammatically, in a sectional view, a portion of a syringe container having external stop portions and internal lock portions; and

FIG. 6 diagrammatically in two sectional views a) and b) show stop and lock arrangements, respectively.

### BRIEF DESCRIPTION OF A PREFERRED EMBODIMENT

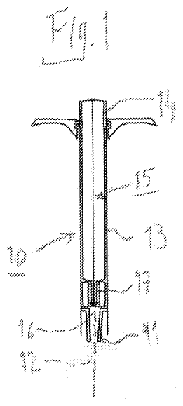

According to preferred embodiments a syringe 10 according to the present invention, FIG. 1, is provided with a front portion 11 carrying a hollow, axially extending needle 12, diagrammatically shown in FIG. 1, and a container 13 or barrel 13, running from said front portion and forming a container rear end portion 14.

The syringe further comprises a plunger 15, eg. FIG. 1, extending and being movable axially in the container, the needle rear end 16 being in fluid connection with the container and the plunger being arranged to suck liquid, e.g. a liquid comprising insulin, glycagone, a vaccine or other substances, through the needle when drawn away from the needle and to discharge liquid through the needle when pushed towards the needle, the plunger being arranged to be connected to a piston portion 17 turned to the needle rear end, the plunger length in relation to the container length being adapted so that when the syringe is arranged in a delivery unused state a certain portion of the plunger extending beyond the container rear end portion.

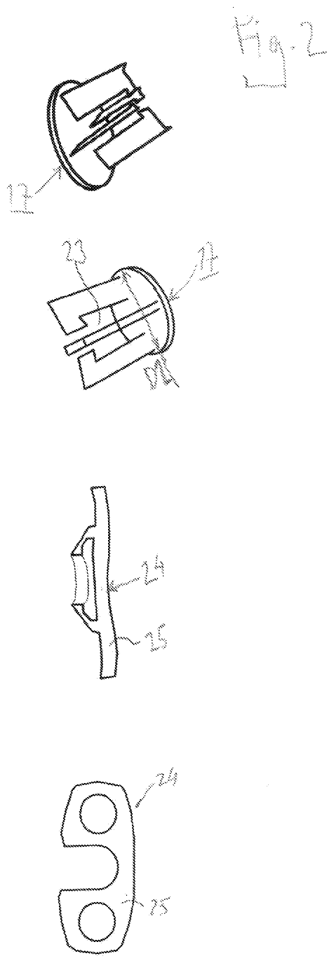

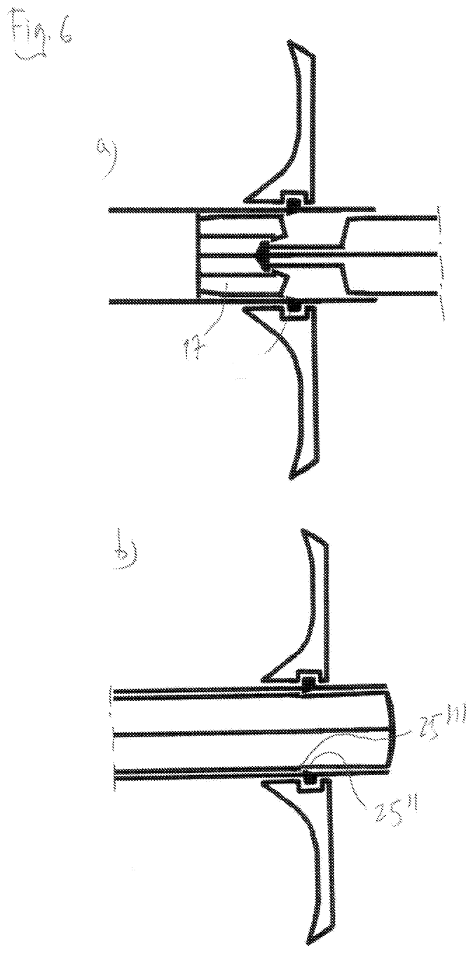

According to the present invention the container wall 18, the barrel wall 18, is made of a cylindrically shaped pipe like body of a material having a certain resilience and an inner diameter D1, FIG. 5, smaller than the outer diameter D2 of the piston portion, FIG. 2, such that the container is locally widened by the piston portion and such that a sealing action appears between the inner wall of the container and the peripheral surface of the piston portion due to a pressure excerted by the widened container wall.

According to preferred embodiments the container is made of a polymeric material, so as to obtain a desired resilience and sealing properties of the container wall. Preferred container material is polypropylene. A preferred material for the plunger, and preferably as well as for the piston portion, is polyethylene.

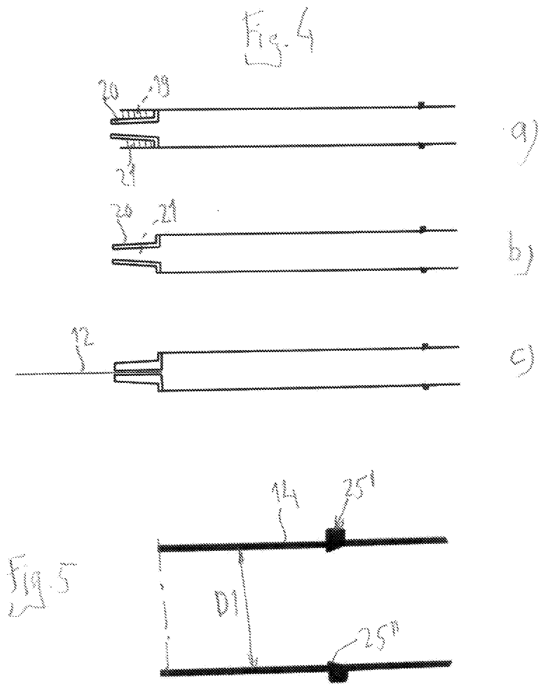

Further according to preferred embodiments, FIG. 4, the needle is carried by a connection portion 19 arranged to be connected to a container front portion 20 by means of a liquid tight thread arrangement 21, FIG. 4a, a liquid tight friction arrangement 21, FIG. 4b) or a fixed needle arrangement FIG. 4c).

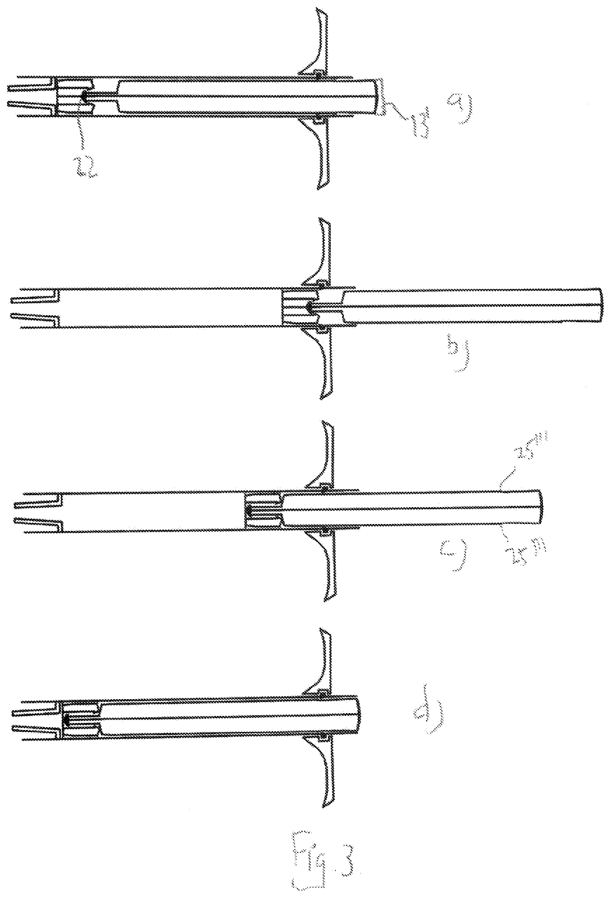

Further the plunger has a front connection portion 22, FIG. 3a), arranged to fit into a rear opening 23, FIG. 2, of the piston portion for connection.

Still further the plunger preferably has a cross like cross section.

Further, according to preferred embodiments, the syringe comprises a grip portion 24, FIG. 2, having extensions 25 for holding the syringe during injection by the syringe, the grip portion preferably being fastened onto the piston by a snap arrangement comprising an outer peripheral stop portion 25′ arranged onto the container at a rear portion, FIG. 5, and, preferably in association with the peripheral outer stop portion 25′, an internal lock arrangement 25″.

FIG. 6a) shows a stop action by means of which the plunger may be prevented from being drawn out of the container by means of coaction between the peripheral stop portion 25′ and piston rear portions.

FIG. 6b) shows a lock action by means of which the plunger is locked against the internal lock arrangement.

According to preferred embodiments the rear end of the plunger comprises a, preferably circular, end plate 13′, FIG. 3a), for moving the plunger in the container and to be concealed in the end of the container during a locked state of the syringe.

The function of the syringe according to the present invention should to a considerable and sufficient extent have been made clear by the description given above.

Thus, in a delivery state, i.e an unused state, FIG. 3a), the syringe is arranged with the piston portion in a front position in the container and the rear end of the plunger being extending a certain distance beyond the rear end of the container, and carrying said grip.

To fill the syringe the needle is introduced into a liquid, e.g. a liquid comprising insulin, glycagon, a vaccine or other substances, the plunger is pulled backwards in the container, whereby the liquid is sucked into the container through the needle by the piston portion drawn by the connection portion 22 to fill the container to a desired extent.

To discharge liquid from the syringe to empty it through the needle, the plunger is pushed forward in the container. The coupling portion is pushed forward to the front position and a locking flange arrangement 25′″ of the plunger is pushed forward, passing the internal lock arrangement 25″ of the container, thereby locking the plunger in the container in a non re-use state of the syringe.

Further, according to preferred embodiments, the container and the plunger are made of polymeric material.

Thus, the present invention is not limited to the embodiments shown but may be varied within the scope of the attached claims.

1-10. (canceled)

11. A syringe comprising:
a front portion carrying a hollow, axially extending needle;
a container running from said front portion and forming a container rear end portion;
a plunger extending and being movable axially in the container;
a needle rear end being in fluid connection with the container;
the plunger being arranged to suck liquid through the needle from the container when drawn away from the needle and to discharge liquid through the needle when pushed towards the needle;
the plunger having a piston portion turned to the needle rear end;
the plunger length in relation to the container length being adapted so that when the syringe is arranged in a delivery state with the piston portion drawn backwards, a certain portion of the plunger extending beyond the container rear end portion;
wherein the container wall is made of a cylindrically shaped pipe like body of a material, having a certain resiliency and an inner diameter (D1) smaller than the outer diameter (D2) of the piston portion;
the container is widened locally by the piston portion; and
a sealing action appears between the inner wall of the container and the peripheral surface of the piston portion due to a pressure exerted by the widened container wall.

12. The syringe according to claim 11, wherein the container is made of a polymeric material to provide a desired resilience and sealing properties of the container wall.

13. The syringe according to claim 11, wherein the needle is carried by a connection portion arranged to be connected to a container front portion by means of a liquid tight thread arrangement or liquid tight friction arrangement or a fixed needle connection extending from the container.

14. The syringe according to claim 11, wherein the plunger has a front connection portion arranged to fit into a rear opening of the piston portion.

15. The syringe according to claim 11, wherein the plunger has a cross like cross section.

16. The syringe according to claim 1, wherein the syringe comprises a grip portion having extensions for holding the syringe during injection by the syringe, the grip portion preferably being fastened onto the piston by a snap arrangement.

17. The syringe according to claim 11, wherein the size of the syringe is adapted to the kind of liquid to be injected such that if a larger volume of liquid is desired a bigger size of the syringe is necessary for obtaining a desired effect of a certain injection.

18. The syringe according to claim 11, wherein it is adapted to contain a single vaccine dose.

19. The syringe according to claim 18, wherein the single vaccine dose is about one milliliter.

20. The syringe according claim 11, wherein the rear end of the plunger comprises an end plate for moving the plunger in the container and to be concealed in the end of the container during a locked state of the syringe.

21. The syringe according claim 10, wherein the end plate is circular.

22. The syringe according to claim 11, wherein a container rear end portion comprises an internal lock arrangement and a plunger portion extending a certain distance beyond the container rear end portion and comprising an outer stop arrangement, so that when the plunger, comprising a connection portion in a discharge state is pushed forward to the front position of the plunger in said container, the locking flange arrangement of the piston portion is pushed forward, passing the internal stop arrangement of the container, thereby locking the plunger in the container in a non re-usable state of the syringe.

23. The syringe according to claim 11, wherein the liquid comprising insulin, glycagon, or a vaccine.
